(19) United States
(12) Patent Application Publication
(10) Pub. No.: US 2010/0286626 A1
(43) Pub. Date: Nov. 11, 2010

(54) Longitudinally incompressible, laterally flexible interior shaft for catheter

(76) Inventors: Scott R. Petersen, Brooklyn Park, MN (US); Dale E. Just, Minneapolis, MN (US)

Correspondence Address: SJM/AFD - DYKEMA;c/o CPA Global, P.O. Box 52050, Minneapolis, MN, 55402, US

(21) Appl. No.: 12/743,631

(22) PCT Filed: Dec. 18, 2008
(86) PCT No.: PCT/US2008/087524

§ 371 Date: May 19, 2010

Related U.S. Application Data
(60) Provisional application No. 61/016,174, filed on Dec. 21, 2007.

(51) Int. Cl. A61M 25/00

(52) U.S. Cl. 604/264

(57) ABSTRACT

A catheter having a substantially longitudinally non-compressible non-metallic internal shaft or reinforcing member (12) is provided having proximal end, intermediate, and distal end portions. At least one of the portions of the shaft includes mechanical features that promote lateral bending of the shaft, and thus, the catheter. In one form, a plurality of spaced-apart cutouts (20,34) are formed within the shaft (12). A first of the plurality of cutouts (20) is disposed in a first plane. A second of the plurality of cutouts (34) is disposed in a second plane. The first plane and second plane can be substantially parallel. The cut-outs (20,34) can have diverse shape, size and cross-section and can be similar, identical and/or of varying dimension. In lieu of or in addition to one or more cut-outs, material removed from portions of the shaft provide relatively more or less flexibility in one or more lateral directions.

### CROSS-REFERENCE TO RELATED APPLICATIONS

This application claims the benefit of and priority to provisional U.S. patent application No. 61/016,174 filed on 21 Dec. 2007 (the '174 application). The '174 application is hereby incorporated by reference as though fully set forth herein.

### BACKGROUND OF THE INVENTION

a. Field of the Invention

This disclosure relates to deflectable medical catheters. More particularly the disclosure relates to a family of substantially non-conductive and longitudinally incompressible elongate members, or shafts, incorporated into a deflectable medical catheter that repeatedly deflects without deformation and/or is compatible with high intensity electromagnetic fields.

b. Background Art

It is well known that devices may be inserted into a body for a variety of diagnostic and therapeutic applications. One example of an application may be to create images of the device's surroundings. Images may be acquired through visible light, ultrasound, or magnetic resonance (MR). A device may therefore be used to acquire high resolution radiographic images of tissue surrounding the device, for example, the acquisition of high resolution magnetic resonance images of blood vessel walls for the visualization and differentiation of various types of tissues and plaques. Another example of an application may be to provide a means to ablate tissue. An imaging or ablating device may be incorporated into a catheter which is placed within a selected blood vessel. A device may therefore be used to acquire high resolution radiographic images of tissue surrounding the device, for example, the acquisition of high resolution magnetic resonance images of blood vessel walls for the visualization and differentiation of various types of tissues and plaques. Another example of an application may be to provide a means to ablate tissue. An imaging or ablating device may be incorporated into a catheter which is placed within a selected blood vessel.

A device generally includes a control system to control a set of tracking coils to sense and indicate the location and orientation of the device within the body, to control a set of imaging coils to image selective areas within the body cavity, and/or to control the amount of energy supplied to the ablation elements to treat target tissues.

Conventional designs for catheters (e.g., steerable ablation catheters or fixed curve catheters) generally provide good shaft torqueability and pushability, while retaining distal flexibility. To achieve these properties, conventional designs include metal braiding, for example, a metal braided tube covered in an outer jacket. However, the use of the metal braiding at the distal end of a catheter may create distortions and/or artifacts in images obtained by a magnetic resonance imaging (MRI) scanner and may not be compatible with MRI scanners, for instance due to undesirable electrical current induced in or through the catheter.

Thus, there remains a need for a shaft member for a deflectable catheter that resists deformation in a longitudinal dimension and/or includes a minimal amount of electrically conductive and/or magnetic material (e.g., metal braiding which may be incompatible with MRI scanners) at least at a distal end portion and yet provides sufficient torqueability, pushability, and flexibility compared with catheter shafts employing metal braiding.

### BRIEF SUMMARY OF THE INVENTION

This disclosure provides a family of substantially non-conductive longitudinally incompressible catheter shafts capable of repeated deflection via application of tension to cables, cords, wires, and the like. Further provided are unique catheter shafts that are essentially indifferent to electromagnetic fields, including high Tesla fields used in MRI as well as radiofrequency radiation. Such catheters withstand repeated computer-controlled deployment and deflection while compatible with MRI scanners. Catheters according to the foregoing are provided herein while also providing adequate torqueability, pushability, and flexibility commonly associated with, and desirable attributes of, conventional deflectable catheter designs.

This disclosure is directed toward a family of substantially non-conductive and longitudinally incompressible elongate members, or shafts for a medical device, such as a catheter. While embodiments of the invention include the substantially longitudinally incompressible elongate member, at least a portion of the member include surface features that promote simple, compound and/or complex lateral bending, or “steering,” of the catheter (e.g., bi-direction, uni-directional, omni-directional and/or planar bending). These surface features can be situated on a proximal end portion, an intermediate portion, and/or a distal end portion and the deflection can be accomplished using adjustable tensioning cords or the like secured at various locations within the catheter. The cords can comprise one or more filaments (e.g., Kevlar® filaments) or the like.

A catheter having a non-metal shaft is provided that is characterized as MRI-compatible in that the catheter is free of materials that can cause image artifacts and/or distortion in an MR image and also does not cause electrical current to be induced in the catheter. The shaft has a proximal end and a distal end. According to one embodiment, one or more portions of the shaft (e.g., the distal end portion) includes a plurality of spaced cutouts, wherein a first of the plurality of cutouts is disposed in a first plane and a second of the plurality of cutouts is disposed in a second plane. The first plane is substantially parallel to the second plane.

The shaft may provide a preferred uniplanar bending pattern along one or more portions of the shaft (e.g., at the distal end portion of the shaft).

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a perspective view of a shaft in accordance with an embodiment of the invention.

FIG. 2 is a cross-sectional view of a catheter employing a shaft in accordance with an embodiment of the invention.

FIG. 2A is a cross-sectional view of a cable conduit that may extend through the shaft in accordance with an embodiment of the invention.

FIG. 3 is an enlarged view of a portion of the shaft of FIG. 1.

FIG. 4 is a perspective view of a shaft in accordance with an embodiment of the invention.

FIG. 5A is a cross-sectional view of a shaft in accordance with an embodiment of the invention.

FIG. 5B is a cross-sectional view of a shaft in accordance with an embodiment of the invention.

FIG. 6A is a cross-sectional view of a shaft in accordance with an embodiment of the invention.

FIG. 6B is a cross-sectional view of a shaft in accordance with an embodiment of the invention.

FIG. 7 is a cross-sectional view of a shaft in accordance with an embodiment of the invention.

### DETAILED DESCRIPTION OF THE INVENTION

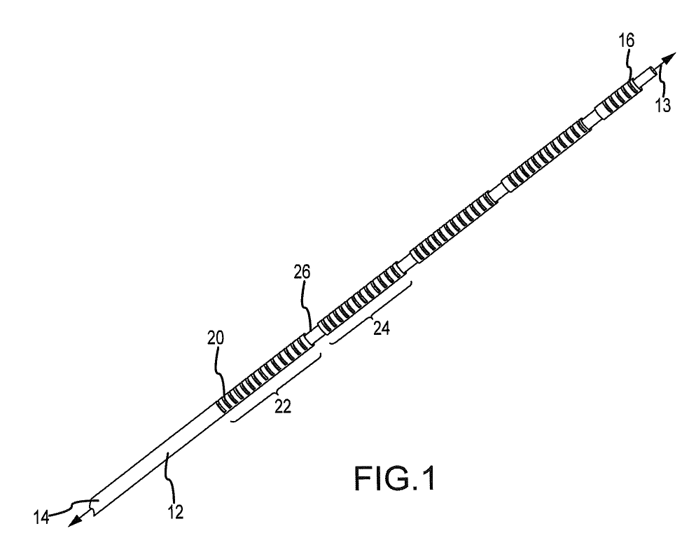

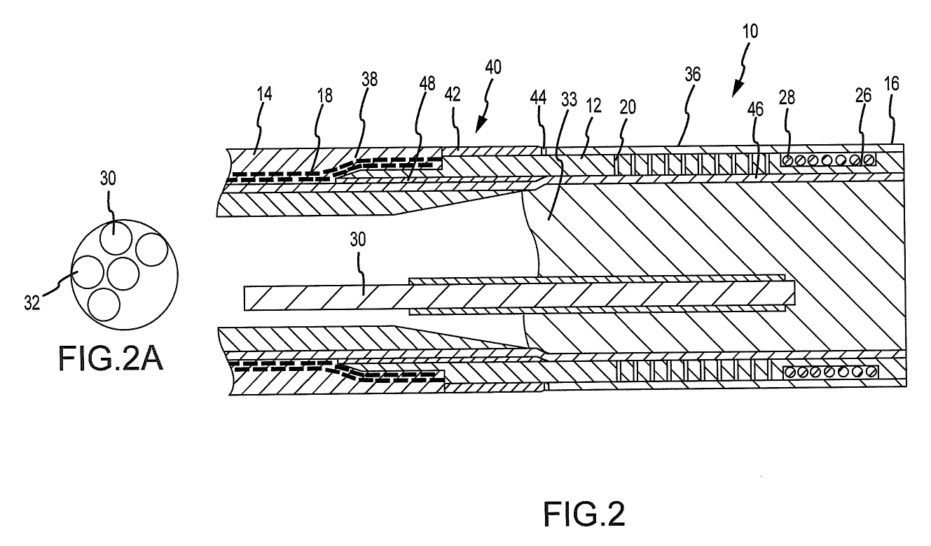

FIG. 1 illustrates a perspective view of a substantially longitudinally incompressible elongate member or shaft 12 in accordance with an embodiment of the invention. Shaft 12 is adapted to be incorporated into a medical catheter device 10 as illustrated in FIG. 2 in accordance with an embodiment of the invention. Shaft 12 is constructed of a substantially non-electrically conductive material (e.g., it can be formed of a non-metallic and/or can comprise a resin-based polymer). In an embodiment, shaft 12 may comprise polyetheretherketones (PEEK) or polyimides. In another embodiment, shaft 12 may comprise a high tensile, high modulus engineering plastic. In yet another embodiment, shaft 12 may comprise a rigid material, such as a rigid plastic or any other of a vast number of resin-based materials. Although these polymers are specifically mentioned, any of various non-metal (e.g., polymeric) materials may be utilized for shaft 12. Shaft 12 may be hollow or can have several longitudinal lumens extending partially or fully therethrough. In an embodiment, shaft 12 may, for example, range from approximately 0.01 inches to 0.10 inches in diameteror approximately 0.0748 inches in diameter. The diameter of shaft 12 may be any of various other diameters. The shaft 12 may extend partially, intermittently, or substantially the full length of a catheter device 10 (as shown in FIG. 2). Shaft 12 include a longitudinal axis (depicted by arrow 13) extending from opposing ends of the shaft, a proximal end portion 14, a distal end portion 16, and an intermediate portion disposed therebetween. Proximal end portion 14 of shaft 12 may remain substantially rigid because of the rigidity of the material used for shaft 12 (e.g., a rigid polymer). Proximal end 14 of shaft 12 may further include metal braiding 18 as illustrated in FIG. 2. Metal braiding 18 may comprise BeCu (beryllium copper) braiding or NiTi (nickel titanium) braiding. Numerous other metals may be used for braiding 18.

Although proximal end portion 14 may be generally rigid, certain portions of a catheter require flexibility. For example, at a steerable region of the catheter on the distal end portion 16, a certain level of flexibility may be desirable or required. Shaft 12 may be configured to provide the desired flexibility at distal end portion 16. For example, in an embodiment, the distal end portion 16 of shaft 12 may be configured to substantially form a roughly ¼ to 2 inch diameter ring or hoop. In some embodiments, the distal end 16 of shaft 12 may be configured to substantially form a roughly ⅝ to ¾ inch diameter ring or hoop. In accordance with an embodiment of the invention, distal end 16 of shaft 12 may be devoid of metal braiding, and may yet be configured to provide similar shaft performance to the distal end of conventional catheter devices with metal braiding. Accordingly, substantially longitudinally incompressible shaft 12 supports and enhances torqueability and pushability to the catheter 10. Of course, the shaft 12 can form part of a pre-curved catheter in addition to or in lieu of a steerable catheter 10 having one or more internal tensioning members coupling the proximal end portion to one or more anchor points (rings and the like) as is known in the art. In an embodiment, the tensioning members are non-conductive elongate members.

At least a portion of the flexibility for distal end portion 16 of shaft 12 may be provided or generated by modifying portions of a shaft. For example, in one embodiment, shaft 12 may have at least a portion of material removed. In an embodiment, material may be completely removed from shaft 12 at select or predetermined radial locations. Shaft 12 may also be formed with the necessary configuration to provide for flexibility at distal end portion 16, rather than having at least a portion of material removed. A formed shaft may include a substantially similar configuration as the modified shaft described (e.g., the formed shaft may include cutouts corresponding to the cutouts described in the modified shaft).

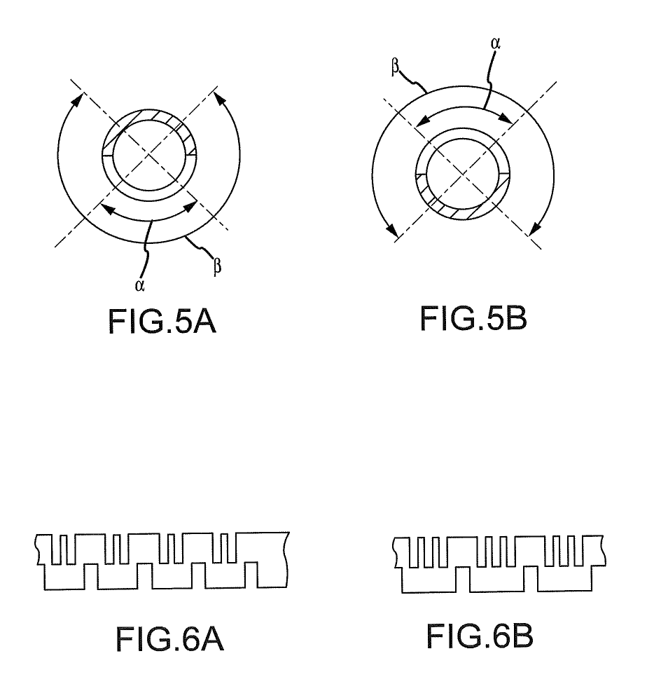

For example, material may be completely removed from shaft 12 through the use of cut-outs 20. Cutouts 20 may be spaced along the longitudinal axis 13 of shaft 12. Cutouts 20 may comprise slots, slits, or curves having the same, similar, and/or different shapes and sizes, for example. Cutouts 20 may extend through a first portion of shaft 12 (e.g., around approximately 180° of an outer circumference of shaft 12). Cutout 20 may extend fewer or more degrees around the outer circumference of shaft 12 in other embodiments. Referring now to FIGS. 5A-5B cutouts 20 may extend around approximately 90° (e.g., angle α) to approximately 270° (e.g., angle β) of an outer circumference of shaft 12. In another embodiment, material may be partially removed to reduce wall thickness of shaft 12 at select or predetermined locations. Material may, for example, be removed from shaft 12 via laser cutting techniques or other mechanical cutting or turning techniques. Although these techniques are specifically mentioned, it is understood by those of ordinary skill in the art that numerous other types of techniques may be used to remove material from shaft 12 and remain within the scope of the invention.

Returning now to FIG. 1, a first series 22 of cutouts 20 may be spaced from an adjacent series 24 of cutouts 20 on shaft 12. The space 26 between a first and second series 22, 24 of cutouts 20 may have a reduced outer diameter compared to other portions of shaft 12. The reduced diameter may be created by the removal of material at the outer surface of shaft 12. Referring now to FIG. 2, a tracking coil 28 may be fully or partially disposed within space 26. Tracking coil 28 may be generally provided in situ on shaft 12 or may be adhered to shaft 12 in any manner conventional in the art. In an embodiment, tracking coil 28 may comprise copper, although other materials may also be employed. Among other things, tracking coil 28 may serve as a tracking mechanism to track the location of distal end 16 of shaft 12 within the body. In an embodiment, tracking coil 28 may be configured to sense a magnetic field, which may be processed in connection with other information to compute the location and orientation of a catheter device within the body. For example, tracking coil 28 may be configured substantially similar to the tracking coils described in U.S. Pat. No. 7,155,271, titled, “System and Method for Magnetic-Resonance-Guided Electrophysiologic and Ablation Procedures,” U.S. Pat. No. 6,246,896, titled “MRI Guided Ablation System,” U.S. Pat. No. 6,275,721, titled “Interactive MRI Scan Control Using an In-Bore Scan Control Device,” and U.S. Pat. No. 6,289,233, titled “High Speed Tracking of Interventional Devices Using an MRI System,” which are hereby incorporated by reference in their entirety as though fully set forth herein. A cable 30 may run through shaft 12 and may be configured to track the location of a catheter device disposed within a body. Cable 30 may be used in connection with tracking coil 28 to provide location information for a catheter device. In an embodiment, cable 30 may comprise a 46 Awg coaxial cable. In an embodiment, a plurality of coaxial cables 30 may extend through a cable conduit 32, for example, as illustrated in FIG. 2A. Cable conduit 32 may, for example, comprise an etched polytetrafluoroethylene (“PTFE”) tube. In an embodiment, five coaxial cables 30 may extend through cable conduit 32. Although the type and number of cables and the cable conduit are specifically mentioned, other types and number of cables and other cable conduits can be used in other embodiments. In accordance with an embodiment of the invention, the remainder of the inside of shaft 12 may comprise a room temperature vulcanizing (RTV) fill material 33 (e.g., RTV silicone fill material).

Variable flexibility at distal end 16 of shaft 12 may be created by changing the quantity of cut-out or removed material. For example, in an embodiment, material may be cut out or removed in only a series of parallel planes (i.e., multiple cutouts 20 in parallel planes along longitudinal axis 13 of shaft 12). Accordingly, flexibility or deflection or movement of the catheter may be provided in a single plane (e.g., preferential uniplanar bending). Deflection in a plane perpendicular (e.g., transverse) to the plane or planes of cut-out or removed material may be resisted. Shaft 12 may therefore allow for uniplanar bending with resistance to bending perpendicular (e.g., transverse) to the plane or planes of the cut-out or removed material. A first cutout 20 may be disposed in a first plane and a second cutout 20 may be disposed in a second plane. The first plane may be substantially parallel to the second plane in an embodiment. The first plane and the second plane may also be substantially non-parallel with longitudinal axis 13 of shaft 12 in an embodiment.

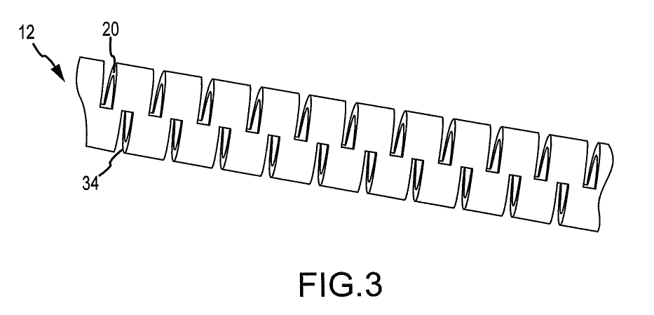

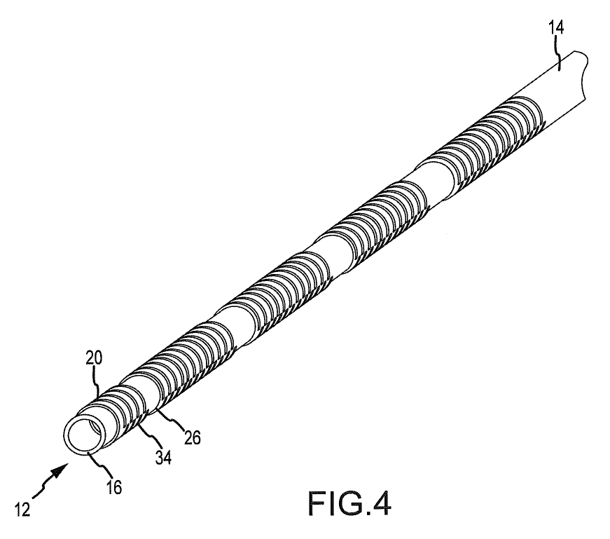

Referring now to FIGS. 3 and 4, material may be cut-out or removed in a series of parallel planes on opposing sides of shaft 12. For example, cutouts 20 may extend through a first portion of shaft 12 (e.g., through approximately a first 180° of an outer circumference of shaft 12), and cutouts 34 may extend through a second portion of shaft 12, the second portion of shaft 12 opposing the first portion (e.g., through approximately a second 180° of an outer circumference of shaft 12). A first cutout 34 may be disposed in a first plane and a second cutout 34 may be disposed in a second plane. The first plane may be substantially parallel to the second plane in an embodiment. The first plane and the second plane may also be substantially non-parallel with longitudinal axis 13 of shaft 12 in an embodiment. Cutouts 20 and cutouts 34 may be in different planes, although these planes may be parallel. Flexibility of shaft 12 may be promoted in a single plane through use of cutouts 20, 34 and may provide resistance to twisting or torsion of shaft 12. As illustrated in FIGS. 3 and 4, cutouts 20 may be offset or staggered from cutouts 34. Although the cutouts 20, 34 are illustrated as offset or staggered which may provide for improved uniplanar bending patterns, cutouts 20,34 may not be offset or staggered in other embodiments.

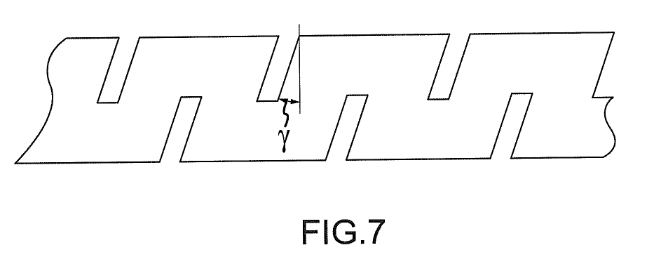

Although the cutouts 20,34 are illustrated in a one-to-one staggered pattern in FIGS. 3 and 4, a two-to-one staggered pattern, a three-to-one staggered pattern, or any of various other staggered patterns (e.g., three-to-two, four-to-two, four-to-three, etc.) may be used in other embodiments. FIGS. 6A and 6B generally illustrate additional staggered patterns that may be used, but it is understood that FIGS. 6A and 6B are for illustrative purposes only and numerous other staggered patterns may be used. Furthermore, cutouts 20,34 may vary in width and remain within the scope of the invention. For example, cutouts 20,34 may, for example, range from approximately 0.001 to approximately 0.080 inches in width. Although this range is mentioned in detail, cutouts 20, 34 may be thinner or thicker in other embodiments.

Although only two sets of opposing cutouts 20,34 are illustrated in FIGS. 3 and 4, additional sets of cutouts or other material removal may be utilized in shaft 12 to affect bending and flexibility in other embodiments. For example, in some embodiments, additional material may be removed in a random pattern throughout the shaft 12 to increase overall or omnidirectional steering flexibility. Such additional material removal is contemplated and is understood by one of ordinary skill in the art to be within the scope of the invention.

The shape or geometry of the cutouts or removed material may also affect the flexibility of shaft 12. For example, cutouts 20,34 may be generally square or rectangular in shape as illustrated in FIG. 3. However, cutouts 20,34 may be wedge shaped, triangular shaped, curved, or any of numerous other shapes in some embodiments. For another example, cutouts 20,34 may be generally perpendicular to longitudinal axis 13 of shaft 12. However, cutouts 20, 34 may not be generally perpendicular to longitudinal axis 13 of shaft 12 in other embodiments. FIG. 7 generally illustrates cutouts that are not perpendicular to longitudinal axis 13 and are offset by angle γ. It is understood that FIG. 7 is for illustrative purposes only and numerous other orientations for cutouts may be used and remain within the scope of the invention. Differences in the shape or geometry of cutouts 20,34 may affect the circumferential interference or coincidence of shaft 12 as is recognized by those of ordinary skill in the art.

The frequency of the cut-out or removed material may also affect the flexibility of distal end 16 of shaft 12. That is, the spacing between cut-out or removed material may be varied and/or variable in order to affect flexibility of the catheter at different points along the longitudinal axis of the catheter. Cut-out or removed material that is spaced closer together along the longitudinal axis may provide increased flexibility (and for some applications, a tighter local radius), while cut-out or removed material that is spaced farther apart along the longitudinal axis may provide decreased flexibility and have a longer radius of curvature given the same magnitude of steering force. In an embodiment, parallel spaced cutouts 20,34 may range from approximately 0.001 to approximately 0.250 inches apart. In an embodiment, parallel spaced cutouts may be approximately 0.0608 inches apart. Although these dimensions are mentioned in detail, cutouts 20 and/or cutouts 34 may be spaced closer or farther apart in other embodiments.

In an embodiment depicted in FIG. 2, the distal end portion 16 of shaft 12 may be partially or substantially fully coated or covered (e.g., encapsulated) with a material 36. Material 36 may comprise a polymer in an embodiment. Material 36 may comprise a low durometer polymer (e.g., durometer level of about 25-40 shore D) to allow for flexibility and bending. For example, without limitation, material 36 may comprise a polyurethane (e.g., a polyurethane sold under the trademark ELASTEON™). Although this material and durometer level is mentioned in detail, material 36 may comprise any number of other materials or may comprise a higher or lower durometer material in other embodiments. Also as depicted in FIG. 2, the proximal end portion 14 of shaft 12 may be partially or substantially fully coated or covered (e.g., encapsulated) with a material 38. Material 38 may comprise a polymer in an embodiment. For example, without limitation, material 38 may comprise a higher durometer polymer than material 36 to allow for further stiffening or reinforcement for proximal end 14 of shaft 12. Material 36 may comprise nylon (e.g., nylon 11) and either or both materials 36,38 may be reflowed over shaft 12. In other embodiments, materials 36,38 may also be epoxied or adhered to shaft 12, or a sleeve of material 36,38 may be placed over and bonded to shaft 12. Although these processes are specifically mentioned, it is understood by those of ordinary skill in the art that any of numerous process may be used to encapsulate shaft 12 with a polymer material and remain within the scope of the invention.

With continuing reference to FIG. 2, in an embodiment of the invention, proximal end 14 and distal end 16 of shaft 12 may be joined together at joint 40. A tube 42 may extend around joint 40. Tube 42 may comprise polyether block amides such as those sold under the trademark PEBAX® and generally available from Arkema France. Tube 42 may comprise 65d PEBAX®. A ring 44 may extend around joint 40. Ring 44 may comprise UVA (e.g., ultra violet adhesive and/or ultra violate cured adhesive).

An inner surface of shaft 12 at distal end 16 may include a lining 46. Lining 46 may substantially extend from distal end 16 through joint 40 to a proximal end 14 of shaft 12. Lining 46 may comprise an aramid resin such as those sold under the trademark Kevlar® and generally available from E. I. du Pont de Nemours and Company. An epoxy 48 may be used at joint 40 to join distal end 16 to proximal end 14.

Although several embodiments of this invention have been described above with a certain degree of particularity, those skilled in the art could make numerous alterations to the disclosed embodiments without departing from the scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the scope of the invention as defined in the appended claims.

1. A catheter having a non-metal internal shaft, said non-metal internal shaft comprising:
a proximal end portion;
an intermediate portion; and
a distal end portion;
wherein said proximal end, intermediate, and distal end portions are each substantially longitudinally incompressible,
wherein at least one of said proximal end, intermediate, and distal end portions includes a plurality of spaced apart flexibility enhancing features, and wherein said features comprise at least one of: a plurality of cutouts and at least one radially-disposed region of reduced thickness, wherein a first set of said features are disposed in a first plane and a second set of said features are disposed in a second plane, and
wherein the first plane is substantially parallel to the second plane.

2. A catheter in accordance with claim 1, wherein the shaft comprises a hollow member.

3. A catheter in accordance with claim 1, wherein the first plane and the second plane are non-parallel with the longitudinal axis of the shaft.

4. (canceled)

5. (canceled)

6. A catheter in accordance with claim 1, wherein the proximal end portion of the shaft includes metal braiding.

7. A catheter in accordance with claim 1, wherein the distal end portion of the shaft is configured to form a curve or arc.

8. A catheter in accordance with claim 7, wherein the distal end portion of the shaft is devoid of any metal material.

9. A catheter in accordance with claim 1, wherein at least one of the first set of features extends approximately 180° around an outer circumference of the shaft.

10. A catheter in accordance with claim 1, further comprising a tracking coil coupled to the outer surface of the shaft.

11. A catheter in accordance with claim 10, wherein the tracking coil couples to the radially-disposed region of reduced thickness.

12. A catheter in accordance with claim 10, wherein the shaft is devoid of cutouts where the tracking coil couples to the shaft.

13. A catheter in accordance with claim 9, wherein the second set of features extends approximately 180° around an outer circumference of the shaft.

14. A catheter in accordance with claim 13, wherein the first set of features is offset from the second set of features.

15. (canceled)

16. A catheter in accordance with claim 1, wherein at least one of the first set of features and the second set of features are generally rectangular in shape.

17. A catheter in accordance with claim 1, wherein at least one of the first set of features and the second set of features are generally wedge shaped.

18. A catheter in accordance with claim 1, wherein at least one of the first set of features and the second set of features is generally irregular in shape.

19. (canceled)

20. A catheter in accordance with claim 1, wherein the distance between the first set of features and the second set of features along a longitudinal axis of the shaft is decreased in order to provide increased flexibility to the distal end.

21. (canceled)

22. (canceled)

23. A catheter in accordance with claim 22, wherein the proximal end portion of the shaft is encapsulated with a second polymer.

24. (canceled)

25. A catheter comprising:
a substantially electrically non-conductive and longitudinally incompressible elongate member;
a plurality of spaced apart structural flexibility enhancing features disposed on laterally opposing sides of the elongate member; and
an outer covering of a biocompatible material surrounding the elongate member;
wherein the flexibility enhancing features comprise at least one of:
a plurality of cutouts and at least one radially-disposed region of reduced thickness,
wherein a first set of features are disposed in a first plane and a second set of features are disposed in a second plane, and
wherein the first plane is substantially parallel to the second plane.

26. A catheter in accordance with claim 25, wherein the elongate member comprises a hollow member and wherein at least a majority of the first set of features and the second set of features are disposed on a distal end portion of the elongate member.

27. (canceled)

28. (canceled)

29. (canceled)

30. A catheter in accordance with claim 25, wherein the proximal end portion of the elongate member includes metal braiding.

31. (canceled)

32. (canceled)

33. (canceled)

34. (canceled)

35. (canceled)

36. A catheter in accordance with claim 10, wherein the tracking coil comprises a magnetic resonance imaging (MRI) tracking coil configured for transmitting a signal to a magnetic resonance imaging (MRI) system.

37. A catheter in accordance with claim 36, wherein the MRI system is responsive to the signal from the tracking coil to depict a location of the catheter in a patient.
